(19) United States
(12) Patent Application Publication
(10) Pub. No.: US 2025/0050004 A1
(43) Pub. Date: Feb. 13, 2025

(54) Pressure Output Device for Extracorporeal Hemodialysis Machine

(71) Applicant: Fresenius Medical Care Holdings, Inc., Waltham, MA (US)

(72) Inventors: James M. Brugger, Newburyport, MA (US); Peter M. Czechowicz, JR., Hudson (OM)

(21) Appl. No.: 18/447,780

(22) Filed: Aug. 10, 2023

(51) Int. Cl. A61M 1/36

(52) U.S. Cl. CPC A61M 1/3621; A61M 2205/3331

(57) ABSTRACT

A pressure output device (POD) for sensing fluid pressure in a blood line set includes a base including an inlet port and an outlet port. The POD further includes a cap coupled to the base to form an interior chamber of the POD, the cap including an outer shell, a pressure sensor port extending from the outer shell, and a tab extending inwardly from an inner surface of the outer shell. The POD also includes a flexible diaphragm coupled to the cap and positioned over the base to divide the interior chamber into a fluid chamber and a pressure sensing chamber, the tab configured to deform a portion of the flexible diaphragm.

### TECHNICAL FIELD

This disclosure relates pressure output devices for measuring fluid pressure in an extracorporeal dialysis machine.

### BACKGROUND

Dialysis is often prescribed for patients who are unable to clear their blood properly using their renal systems (e.g., kidneys).

The two principal dialysis methods are hemodialysis and peritoneal dialysis. During hemodialysis (“HD”), the patient's blood is passed through a dialyzer of a dialysis machine while also passing a dialysis solution or dialysate through the dialyzer. A semi-permeable membrane in the dialyzer separates the blood from the dialysate within the dialyzer and allows diffusion and osmosis exchanges to take place between the dialysate and the blood stream across the membrane. These exchanges across the membrane result in the removal of waste products, including solutes like urea and creatinine, from the blood. These exchanges also regulate the levels of other substances, such as sodium and water, in the blood. In this way, the dialysis machine acts as an artificial kidney for cleansing the blood.

During peritoneal dialysis (“PD”), the patient's peritoneal cavity is periodically infused with dialysate. The membranous lining of the patient's peritoneum acts as a natural semi-permeable membrane that allows diffusion and osmosis exchanges to take place between the solution and the blood stream. These exchanges across the patient's peritoneum result in the removal of waste products, including solutes like urea and creatinine, from the blood, and regulate the levels of other substances, such as sodium and water, in the blood.

In an HD treatment, a patient is connected to an extracorporeal blood circuit by inserting a venous bloodline and an arterial bloodline and a dialysis machine takes in the blood from the arterial line, and flows the blood past a semipermeable membrane or filter that is permeable to toxins and fluid. On the other side of the filter, dialysate flows in the opposite direction. The dialysate is a combination of acid, water, and other chemicals, the most notable of which is bicarbonate. The length of treatment time and concentrations of chemicals within the dialysate are prescribed by a physician and are inputted into the dialysis machine prior to beginning dialysis. The prescription includes a concentration of bicarbonate, a dialysate flow rate and if applicable a substitution fluid pump rate, and a treatment length, among other parameters and concentrations. Often the dialysate is mixed using fluids that are previously saturated with a specific substance. For example, bicarbonate solution is created by mixing fluid with powder bicarbonate concentrate. That bicarbonate solution can then be used to mix with other saturated solutions to create dialysate.

In hemofiltration, solutes are removed through the blood primarily through convection resultant of the introduction of substitution into the blood circuit. In hemodiafiltration, the underlying mechanics of both hemodialysis and hemofiltration are used, resulting in diffusive and convective clearance. Substitution fluid can be generated as a bolus, or continuously during treatment at a prescribed rate. The resultant true dialysate consumption rate in hemofiltration and hemodiafiltration treatments are the sum total of the dialysate flow rate and the substitution fluid flow rate.

### SUMMARY

In an aspect, a pressure output device (POD) for sensing fluid pressure in a blood line set includes a base including an inlet port and an outlet port. The POD further includes a cap coupled to the base to form an interior chamber of the POD, the cap including an outer shell, a pressure sensor port extending from the outer shell, and a tab extending inwardly from an inner surface of the outer shell. The POD also includes a flexible diaphragm coupled to the cap and positioned over the base to divide the interior chamber into a fluid chamber and a pressure sensing chamber, the tab configured to deform a portion of the flexible diaphragm.

Some embodiments provided herein incorporate one or more of the following features.

In some embodiments, the tab is positioned beneath the pressure sensor port.

In some embodiments, the tab is configured to prevent the flexible diaphragm from covering an opening of the pressure sensor port.

In some embodiments, the cap and the flexible diaphragm are formed using a two shot molding process.

In some embodiments, the tab includes a first portion configured to contact the flexible diaphragm, and a second portion configured to bend to adjust a position of the first portion.

In some embodiments, a width of the first portion is larger than a width of the second portion.

In some embodiments, a thickness of the first portion is larger than a thickness of the second portion.

In some embodiments, the first portion has a cross-sectional shape that is circular, rectangular, elliptical, or square.

In some embodiments, the tab elastically dimples the flexible diaphragm when the tab is in a bent position and the flexible diaphragm contacts the tab.

In some embodiments, the base and the outer shell of the cap are semi-rigid.

In some embodiments, the base and the outer shell of the cap are translucent.

In some embodiments, the pressure sensor port is configured to couple to a luer connector of a blood treatment machine.

In some embodiments, the pressure sensor port is configured to fluidly couple to a pressure sensor of a blood treatment machine.

In some embodiments, the tab is configured to be bent into a bent position prior to a blood treatment performed using the blood line set.

In some embodiments, the tab is configured to be bent into the bent position by a pin of a blood treatment machine that extends through the pressure sensor port and contacts the tab when the POD is coupled to the blood treatment machine.

In another aspect, a method of assembling a pressure output device (POD) includes bending a tab of the first portion into a bent configuration in which the tab is configured to form a dimple in a portion of a flexible diaphragm when the flexible diaphragm is in contact with the tab, the portion of the flexible diaphragm being positioned below a pressure sensor outlet of the POD, and connecting the first portion of the POD with the diaphragm therein to a second portion of the POD.

Some embodiments provided herein incorporate one or more of the following features.

In some embodiments, the method includes forming the first portion using a first shot of a two-shot molding process, and forming the diaphragm using a second shot of the two-shot molding process.

In some embodiments, bending the tab includes bending the tab along a first portion of the tab that is thinner or has a smaller width than a second portion of the tab.

In some embodiments, bending the tab includes bending the tab by inserting a rod through a port of the POD and applying a force to the tab.

In an aspect, a method includes bending a tab of a pressure output device (POD) into a bent configuration in which the tab is configured to form a dimple in a portion of a flexible diaphragm of the POD when the flexible diaphragm is in contact with the tab, the portion of the flexible diaphragm being positioned below a pressure sensor outlet of the POD, and fluidly coupling the pressure sensor outlet of the POD to a pressure sensor.

Some embodiments provided herein incorporate one or more of the following features.

In some embodiments, the method includes flowing blood through a fluid chamber of the POD, and measuring a pressure in a pressure sensing chamber of the POD using a pressure sensor fluidly coupled to the pressure sensing chamber of the POD.

In some embodiments, flowing blood through the fluid chamber of the POD includes fluidly coupling a blood line set to a blood treatment machine, and causing a blood pump of the blood treatment machine to pump blood through the blood line set.

In some embodiments, the pressure sensing chamber of the POD is fluidly coupled to the pressure sensor through a pressure sensor outlet of the POD.

In some embodiments, forming a dimple in the portion of the POD prevents vapor lock of the flexible diaphragm against a cap of the POD.

In some embodiments, bending the tab of the POD includes inserting a pin through the pressure sensor outlet of the POD to contact and apply a force to the tab to bend the tab.

In some embodiments, bending the tab of the POD includes attaching the POD to a connector of a blood treatment device, and causing a pin to extend from the blood treatment device, through the pressure sensor outlet of the POD, and apply a force to the tab to bend the tab.

In some embodiments, the method includes coupling an inlet line of a blood line set to an inlet port of the POD, and coupling an outlet line of the blood line set to an outlet port of the POD.

In an aspect, a blood treatment system includes a blood treatment machine, a blood line set configured to be connected to the blood treatment machine, and a pressure output device (POD) for sensing fluid pressure in the blood line set. The POD includes a base including an inlet port and an outlet port. The POD also includes a cap coupled to the base to form an interior chamber of the POD. The cap includes an outer shell, a pressure sensor port extending from the outer shell, and a tab extending inwardly from an inner surface of the outer shell. The POD further includes a flexible diaphragm coupled to the cap and positioned over the base to divide the interior chamber into a fluid chamber and a pressure sensing chamber, the tab configured to deform a portion of the flexible diaphragm.

Some embodiments provided herein incorporate one or more of the following features.

In some embodiments, the blood treatment machine is a dialysis machine.

In some embodiments, the blood treatment machine is a hemodialysis machine, and the blood treatment system further includes a dialyzer.

In some embodiments, the blood line set includes at least one of an arterial line and a venous line.

In some embodiments, the pressure sensor port is configured to couple to a luer connector of the blood treatment machine.

In some embodiments, the tab is configured to be bent into a bent position by a pin of the blood treatment machine that extends through the pressure sensor port and contacts the tab when the POD is coupled to the blood treatment machine.

In some embodiments, the tab is positioned beneath the pressure sensor port.

In some embodiments, the tab is configured to prevent the flexible diaphragm from covering an opening of the pressure sensor port.

In some embodiments, the pressure sensor port is configured to couple to a pressure monitoring port of the blood treatment machine.

In some embodiments, the cap and the flexible diaphragm are formed using a two shot molding process.

In some embodiments, the tab includes a first portion configured to contact the flexible diaphragm, and a second portion configured to bend to adjust a position of the first portion.

In some embodiments, a width of the first portion is larger than a width of the second portion.

In some embodiments, a thickness of the first portion is larger than a thickness of the second portion.

In some embodiments, the first portion has a cross-sectional shape that is circular, rectangular, elliptical, or square.

In some embodiments, the base and the outer shell of the cap are semi-rigid.

In some embodiments, the base and the outer shell of the cap are translucent.

The details of one or more embodiments are set forth in the accompanying drawings and the description below. Other aspects, features, and advantages of the disclosure will be apparent from the description and drawings, and from the claims.

### DESCRIPTION OF DRAWINGS

FIG. 1 is a schematic perspective view of a pressure output device (POD) of a blood line set.

FIG. 2 is an exploded view of the POD of FIG. 1.

FIG. 3 is a schematic cross-sectional view of the POD of FIG. 1.

FIG. 4 is a schematic top view of an example tab of the POD of FIG. 1.

FIG. 5 is a schematic of an extracorporeal circuit with an arterial side POD and a venous side POD.

FIG. 6 is a detailed view of example devices for connecting PODs to a blood treatment machine.

FIG. 7 is a schematic cross-sectional view of the POD of FIG. 1 connected to a blood treatment machine.

FIG. 8 is schematic perspective view of a device for coupling the POD of FIG. 1 to a blood treatment machine.

FIG. 9 is a flow diagram for an example process of assembling a POD for use in an extracorporeal circuit.

FIG. 10 is a flow diagram for another example process of assembling a POD for use in an extracorporeal circuit.

FIG. 11 is a schematic cross-sectional view of a POD of a blood line set with a tab in an unbent position.

FIG. 12 is a top view of the cap of the POD in FIG. 10.

FIG. 13 is a schematic top view of the tab of the POD of FIG. 10 in an unbent position.

FIGS. 14A and 14B are schematic cross-sectional views of the POD of FIG. 10 connected to a blood treatment machine.

FIGS. 15A and 15B are schematic top views of example tabs for a POD of a blood line set.

FIGS. 16A and 16B are a schematic top view and a schematic side view, respectively, of an example tab for a POD of a blood line set.

### DETAILED DESCRIPTION

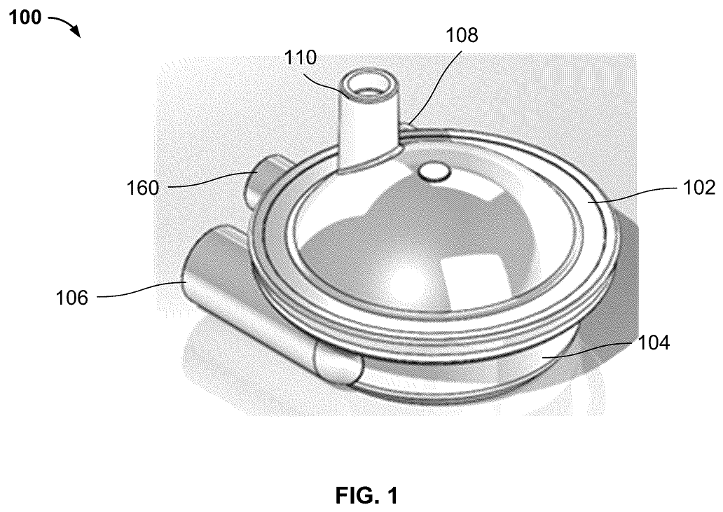

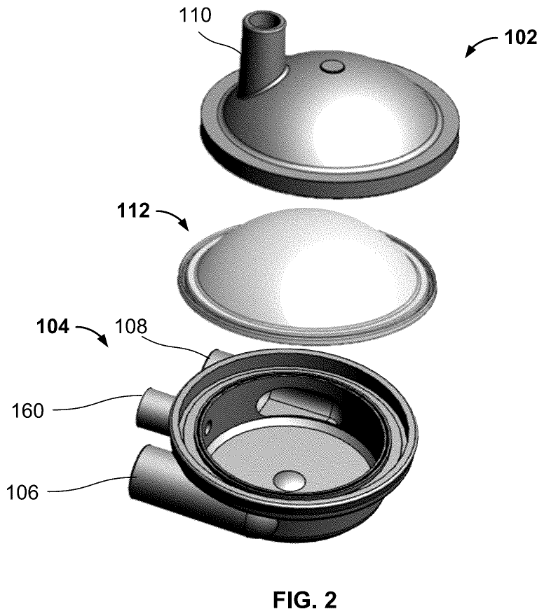

FIG. 1 illustrates an example pressure output device (POD) 100 that can be arranged to measure blood pressure in an arterial circuit. For example, the POD 100 can be placed along and used in arterial and venous lines (collectively referred to as “blood lines”) of an extracorporeal circuit (e.g., of a dialysis machine) for transferring extracorporeal circuit pressures to pressure monitoring ports of the extracorporeal circuit. Referring to FIGS. 1 and 2, the POD 100 includes a cap 102, a base 104, a diaphragm 112 positioned between the cap 102 and the base 104, an inlet port 106, an outlet port 108.

As will be described in further detail herein, a blood line can be connected to the inlet port 106 and the outlet port 108 of the POD 100 in order to allow blood flowing through the blood line to pass through the base 104 of the POD 100 for measurement of pressure in the blood line. A sensor port 110 extends from an outer shell 132 of the cap 102, and the sensor port 110 can be connected to a pressure sensor port of a blood treatment machine (e.g., a hemodialysis machine) to fluidly connect and provide pressure output to the pressure sensor port of the blood treatment machine. For example, the sensor port 110 can fluidly connect to a luer connector of a blood treatment machine or can directly connect to a pressure sensor port of a blood treatment machine in order to provide a pressure output to the blood treatment machine. An injection port 160 of the POD 100 can connect to an injection site and a priming spike to receive, e.g., heparin and fluid, respectively. The injection port 160 can reduce the need for additional connectors from the bloodline assembly that connects to the POD 100.

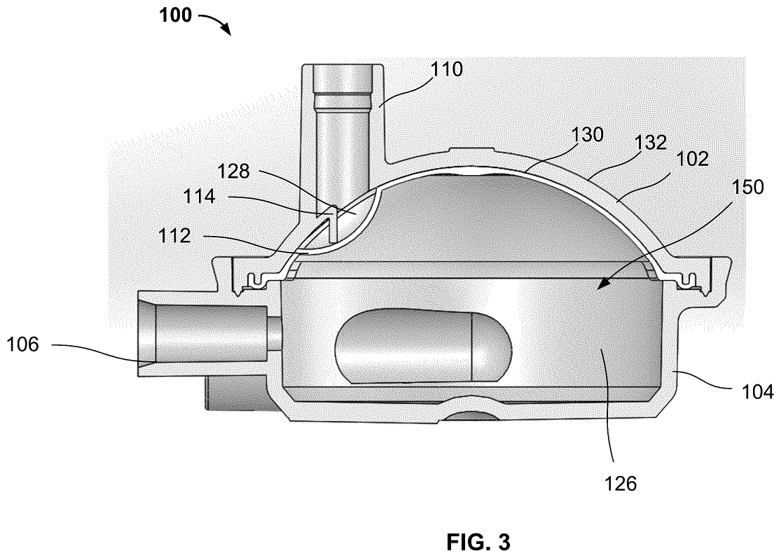

FIG. 3 illustrates a cross-section of the assembled POD 100 of FIG. 1. As can be seen in FIGS. 2 and 3, the cap 102 is attached to the base 104 to form an interior chamber 150 within the POD 100. The diaphragm 112 is attached to the cap 102 and is positioned between the cap 102 and the base 104 to divide the interior chamber 150 into two chambers, a fluid chamber 126 and a pressure sensing chamber 128, in the POD 100. The diaphragm 112 can be formed of any suitable elastomeric material, including, but not limited to, rubber, polyurethane, and silicone. In some implementations, the base 104 and the outer shell 132 of the cap 102 are semi-rigid.

In some implementations, the diaphragm 112 and the cap 102 are formed using a two-shot molding process. Two-shot molding is a subcategory of injection molding that allows multi-material parts to be molded without additional assembly steps. Two-shot molding manufacturing can provide two parts (e.g., the diaphragm 112 and the cap 102 of the POD 100) that are formed together to have the same shape. In two-shot molding, a first material is injected into a mold to create a substrate, around which the other material (or materials in multi-shot molding) will be molded. The substrate solidifies and cools before being transferred (e.g., by hand, robot arm, etc.) to another chamber of the mold. Once the substrate is in place, a second material is injected and bonds with the substrate to form a firm hold. When the second material cools, the second material can be ejected from the substrate. The second material has the same shape as the first material, because the first material is used as a mold for the second material. By using two-shot molding to form the cap 102 of the POD 100 and the diaphragm 112 of the POD 100, the diaphragm 112 is molded within the cap 102 of the POD 100 such that the diaphragm 112 conforms to an inner surface 130 of the outer shell 132 of the cap 102. Using two-shot molding can be advantageous, for example, to prevent air bubbles from becoming trapped within the cap 102 and, as a result, improves the accuracy of the pressure output of the POD 100.

In some implementations, the base 104 and the outer shell 132 of the cap 102 are formed of a semi-rigid material. In some implementations, the base 104 and the outer shell 132 of the cap 102 are formed of a translucent material.

As blood flows through the POD 100, positive or negative circuit pressure displaces the diaphragm 112, and the displacement of the diaphragm 112 causes the volume of air within the pressure sensing chamber 128 between the diaphragm 112 and the pressure sensor and in communication via the sensor port 110 to either compress or expand. As the air volume within the pressure sensing chamber 128 changes, the pressure sensor fluidly coupled to the POD 100 via the sensor port 110 detects the resulting pressure change.

For example, as blood flows from a blood line connected to the POD 100 (e.g., from a venous blood line) into the inlet port 106, the fluid chamber 126 of the POD 100 fills with blood, which exerts a positive pressure on the diaphragm 112 and causes the diaphragm 112 to flex upwards toward the cap 102 to accommodate the blood within the fluid chamber 126. As pressure increases in the blood line coupled to the POD 100 (e.g., due to increased blood pressure in the patient), the pressure within the fluid chamber 126 of the POD 100 increases correspondingly as the higher pressure blood flow enter the inlet port 106, and, as a result, the diaphragm 112 moves closer towards the cap 102. The movement of the diaphragm 112 towards the cap 102 compresses air within the pressure sensing chamber 128 and the sensor port 110, which increases the pressure placed on the pressure sensor fluidly coupled to the POD 100 through connection to the sensor port 110.

In addition, the POD 100 can be used to detect a drop in pressure within a blood line coupled to the POD 100. For example, when pressure decreases within the blood line, the volume of fluid within the fluid chamber 126 of the POD decreases, which causes the diaphragm 112 to move away from the inner surface 130 of the cap 102. The movement of the diaphragm 112 away from the cap 102 causes expansion of the air within the pressure sensing chamber 128, which results in a reduction in pressure placed on the pressure sensor fluidly coupled to the POD 100 through connection to the sensor port 110.

In addition, the POD 100 can be used to measure a negative pressure within a blood line that connected to the POD (e.g., within an arterial blood line) as blood flows through the blood line and into the POD 100. For example, during treatment, negative pressure is generated within the arterial blood line as blood is withdrawn from the patient through the arterial blood line (e.g., using a blood pump). In order to measure changes in negative pressure within the arterial blood line, the POD configured to be coupled to the arterial blood line is primed by providing air into the sensing chamber 128 in order to force the diaphragm 112 downwards towards the fluid chamber 126, which reduces the pressure within the sensing chamber 128 and enables the POD to measure changes in negative fluid pressure within the arterial blood line.

Conversely, positive pressure is generated in the venous blood line during treatment as blood is returned to the patient through the venous line (e.g., using a blood pump). In order to measure changes in the positive pressure within in the venous blood line, the POD configured to be coupled to the venous blood line is primed by providing air into the fluid chamber 126 to force the diaphragm 112 upwards towards the sensing chamber 128, which increases the pressure within the sensing chamber 128 and enables the POD to measure changes in positive fluid pressure within the venous blood line.

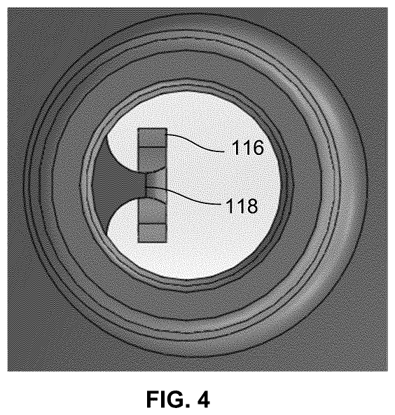

The cap 102 includes a tab 114 that is bent downwards and extends inwardly from the inner surface 130 of the outer shell 132 of the cap 102 to prevent the diaphragm 112 from forming a seal (e.g., vapor lock) over the sensor port 110. The tab 114 is positioned below the sensor port 110 and is bent away from the sensor port 110. FIG. 4 illustrates the tab 114 from above (e.g., as seen through the sensor port 110). The tab 114 includes a head portion 116 that is wider than a hinge portion 118 that connects the head portion 116 to the cap 102. The head portion 116 of the tab 114 is generally elliptical. The hinge portion 118 is deformable so that the hinge portion 118 bends relative to the cap 102. For example, the hinge portion 118 of the tab 114 has a smaller width than the head portion 116 of the tab 114 so that the hinge portion 118 deforms to bend the tab 114 downwards when a downward force is applied to the head portion 116 of the tab 114.

The bent tab 114 of the cap 102 can prevent vapor lock between the diaphragm 112 and the cap 102 when the pressure within a blood line coupled to the POD 100 is high. As previously discussed, the cap 102 and diaphragm 112 are formed using two-shot molding, which results in the diaphragm 112 conforming to the inner surface 130 of the cap 102. As a result, whenever the pressure within a blood line coupled to the POD 100 is sufficiently high, the pressure within the fluid chamber 126 causes the diaphragm 112 to flex towards and conform to the inner surface of the cap 102, as depicted in FIG. 3. As the diaphragm 112 comes into contact with the inner surface of the cap 102, the tab 114 contacts and forms a dimple in the diaphragm 112 (e.g., deform the diaphragm 112 away from the sensor port 110). In particular, as the diaphragm 112 comes into contact with the inner surface of the cap 102, the tab 114 forms a dimple in a portion of the diaphragm 112 positioned below the sensor port 110, as can be seen in FIG. 3, which prevents vapor lock by deforming the diaphragm 112 away from the sensor port 110 and preventing the diaphragm 112 from forming a seal with the sensor port 110. Preventing vapor lock between the diaphragm 112 and the cap 102 using tab 114 allows the POD 100 to accurately measure changes in pressure in a blood line coupled to the POD 100 when the pressure in the blood line is high.

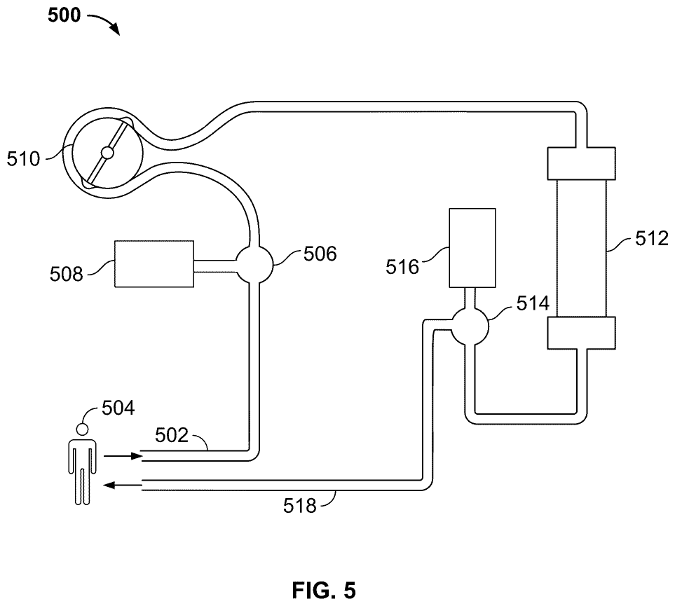

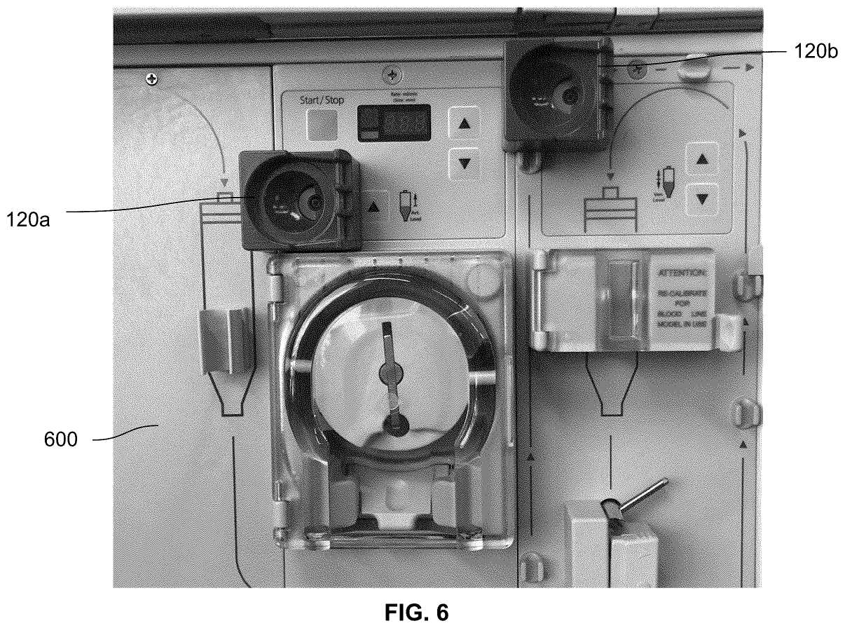

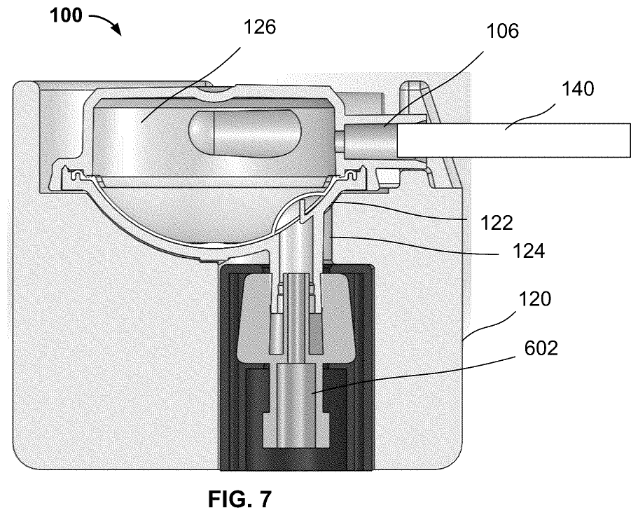

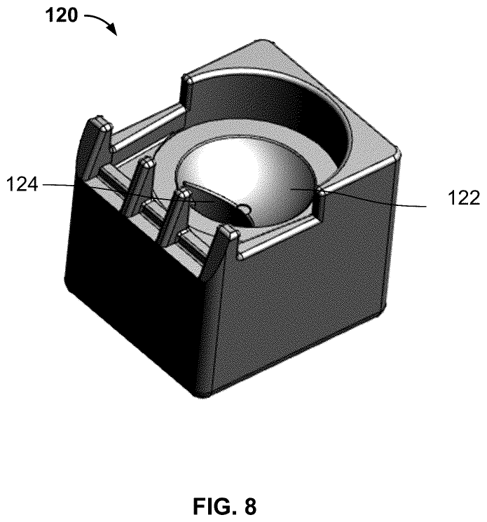

FIG. 5 depicts an example extracorporeal blood circuit 500 for administration of hemodialysis with an arterial POD 506 and a venous POD 514 fluidly coupled to an arterial line 502 and a venous line 518, respectively. As depicted in FIG. 6, in some implementations, the arterial POD 506 and the venous POD 514 are connected to the blood treatment machine 600 using an arterial pressure monitoring port 120a and a venous pressure monitoring port 120b (referred to herein as pressure monitoring port 120), respectively. The blood treatment machine 600 can be fluidly coupled to an extracorporeal blood circuit similar to the blood circuit 500. As depicted in FIGS. 7 and 8, the pressure monitoring port 120 includes a seat 122 shaped to receive the POD 100, and the sensor port 110 of the POD 100 can be received in a recess 124 of the pressure monitoring port 120. When the POD 100 is coupled to the pressure monitoring port 120, the sensor port 110 is positioned within the recess 124 such that the pressure sensor 602 is fluidly connected to the sensor port 110. In some implementations, prior to starting treatment and flowing fluid into the fluid chamber 128 of the POD from a blood line, the pressure sensor 602 fluidly coupled to the POD is “zeroed” such that that the pressure sensor 602 is set to measures no applied pressure while fluid is not being drawn through the POD. In some implementations, the POD 100 is calibrated during a priming sequence of the blood treatment machine 600. In some implementations, the blood treatment machine 600 can compare any pressure differential between an open sensor port (e.g., pressure monitoring ports 120) to the pressure detected once the POD 100 is coupled to the port in order to calibrate the POD 100.

The pressure sensor 602 is configured to detect pressure output by the POD 100. For example, as blood flows through the POD 100, positive or negative pressure within the fluid chamber 126 of the POD 100 displaces the diaphragm 112, and the displacement of the diaphragm 112 causes the volume of air within the pressure sensing chamber 128 between the diaphragm 112 and the pressure sensor 602 to either compress or expand. As the air volume within the pressure sensing chamber 128 changes, the pressure sensor 602 detects the resulting pressure change within the pressure sensing chamber 128. For example, as blood flows from a blood line connected to the POD 100 (e.g., blood lines 502, 518 of FIG. 5) into the inlet port 106, the fluid chamber 126 of the POD 100 fills with blood, which exerts a pressure on and causes the diaphragm 112 to flex upwards toward the cap 102 in order to accommodate the blood within the fluid chamber 126. As pressure increases in the blood circuit, the pressure within the fluid chamber 126 of the POD 100 increases correspondingly as the higher pressure blood flow enters the fluid chamber 126 of the POD 100, and, as a result, the diaphragm 112 moves closer towards the cap 102. The movement of the diaphragm 112 towards the cap 102 compresses air within the pressure sensing chamber 128 and the sensor port 110, which increases the pressure placed on the pressure sensor 602 that is fluidly coupled to the POD 100 through connection to the sensor port 110. Conversely, when pressure decreases within the blood line, the pressure within the fluid chamber 126 of the POD 100 also decreases, which causes the diaphragm 112 to move away from the inner surface 130 of the cap 102 . . . . The movement of the diaphragm 112 away from the cap 102 causes expansion of the air within the pressure sensing chamber 128, which results in a reduction in pressure placed on the pressure sensor 602 that is fluidly coupled to the POD 100 through connection to the sensor port 110

As discussed above, the bent tab 114 of the cap 102 can prevent vapor lock between the diaphragm 112 and the cap 102. The cap 102 and diaphragm 112 are formed using two-shot molding, which results in the diaphragm 112 conforming to the inner surface 130 of the cap 102. As a result, whenever the pressure within a blood line coupled to the POD 100 (e.g., lines 502, 518 of FIG. 5) is sufficiently high, the pressure within the fluid chamber 126 causes the diaphragm 112 to flex towards and conform to the inner surface of the cap 102, as depicted in FIG. 3. As the diaphragm 112 comes into contact with the inner surface of the cap 102, the tab 114 contacts and deforms the diaphragm 112 away from the sensor port 110 (e.g., by dimpling the diaphragm 112), which prevents vapor lock by deforming the diaphragm 112 away from the sensor port 110 and preventing the diaphragm 112 from forming a seal with the sensor port 110.

A process of measuring pressure in a blood lines of extracorporeal circuits using PODs will be described in reference to FIGS. 5-8. An arterial line 502 carries blood from a patient 504 to an arterial POD 506 (e.g., similar to the POD 100 of FIG. 1). An arterial pressure sensor 508 of a blood treatment machine is connected to the sensor port of the POD 506 (e.g., similar to sensor port 110 of FIG. 1) and receives a pressure output from the arterial POD 506 that represents the pressure within the arterial line 502 connected the arterial POD 506. The POD 100 is operatively connected to a pressure sensor 602 of the blood treatment machine using the arterial pressure monitoring port 120a of the blood treatment machine 600. As depicted in FIG. 7, a blood line 140 (e.g., arterial line 502 or venous line 518 of FIG. 5) can be provided in fluid communication with the fluid chamber 126 of the POD 100 through connection of the fluid line to the inlet port 106 and the outlet port 108 of the POD 100 and can be operatively positioned with respect to the pump 510 of the blood treatment machine 600 such that the pump 510 moves fluid through the blood line 140 and through the fluid chamber 126 of the POD 100. In some cases, the pump 510 is a peristaltic pump and the fluid line is positioned so as to be acted upon by a rotor of the peristaltic pump, for example, in a raceway or semi-circular track.

As blood flows through from the arterial line 502 into the fluid chamber of the arterial POD 506, a pressure sensor of the blood treatment machine 600 fluidly coupled to the arterial POD 506 is configured to detect pressure changes resulting from movement of the diaphragm of the arterial POD 506. For example, the pressure sensor can be configured for sensing pulses of pressure caused by the pump 510 moving fluid through the fluid chamber of the arterial POD 506. Similarly, as blood flows through from the venous line 518 into the fluid chamber of the venous POD 514, a pressure sensor of the blood treatment machine 600 that is fluidly coupled to the venous POD 514 is configured to detect pressure changes resulting from movement of the diaphragm of the venous POD 514. For example, the pressure sensor can be configured for sensing pulses of pressure caused by the pump 510 moving fluid through the fluid chamber of the venous POD 514.

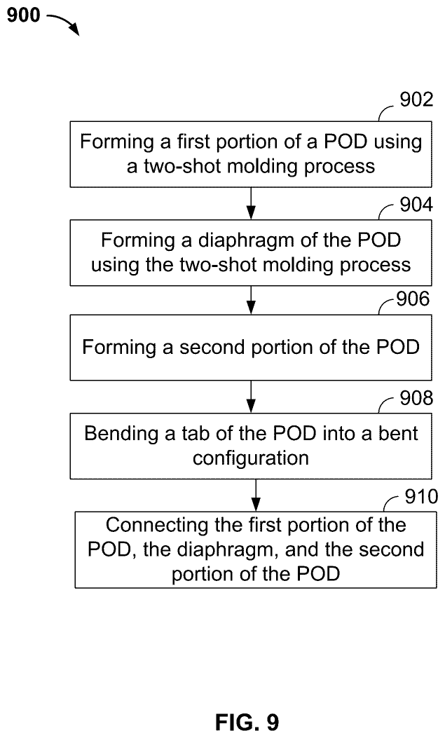

FIG. 9 is a flowchart of an example method 900 of manufacturing a POD having a tab that is bent (e.g., similar to the POD 100 of FIG. 1). A first portion of the POD is manufactured using the first shot of a two-shot molding process (902) and a diaphragm of the POD (e.g., diaphragm 112 of FIG. 3) is formed using the second shot of the two-shot molding process (904). The first portion of the POD is, e.g., the cap (e.g., cap 102 of FIGS. 1-3).

Two-shot molding is a subcategory of injection molding that allows multi-material parts to be molded without additional assembly steps. Using two-shot molding provides two parts (e.g., two portions of a POD) that fit together properly. In two-shot molding, a first material is injected into a mold to create a substrate, around which the other material (or materials in multi-shot molding) will be molded. The substrate solidifies and cools before being transferred (e.g., by hand, robot arm, etc.) to another chamber of the mold. Once the substrate is in place, a second material is injected and bonds with the substrate to form a firm hold. When the second material cools, the second material can be ejected from the substrate.

By using two-shot molding to form the first portion of the POD and the diaphragm of the POD, the diaphragm is molded within the first portion of the POD so that the diaphragm fits properly within the first portion of the POD. Molding the diaphragm within the first portion of the POD can be advantageous, for example, to prevent air bubbles from becoming trapped within the POD and, as a result, improves the accuracy of the pressure output of the POD. The first portion of the POD is injection molded, e.g., using acrylonitrile butadiene styrene (ABS), another thermoplastic material such as polycarbonate, or the like.

A second portion of the POD is formed (906). For example, forming the second portion of the POD includes multi-shot molding, as described above. In some implementations, the second portion of the POD is injection molded using a thermoplastic material such as ABS, polycarbonate, or any other suitable thermoplastic material. In some implementations, the first portion and second portion of the POD are semi-rigid.

A tab of the POD (e.g., tab 114 of FIGS. 3 and 4) is bent into a bent configuration (908). For example, the tab is integrally molded with the first portion of the POD, and the tab is bent along a hinge of the tab (e.g., a portion of the tab that is thinner and/or has a smaller width than another portion of the tab). In some implementations, the tab is bent by an operator involved in the manufacturing process. For example, an operator manually bends the tab, e.g., by inserting a rod through the sensor port of the POD to apply a downward force to a portion of the POD (e.g., to the head portion 116 of tab 114). In some implementations, the operator manually bends the tab by grasping and bending the tab using her fingers. In some implementations, the tab is bent automatically during the manufacturing process. For example, after forming the first portion and diaphragm of the POD, the injection molding machine automatically bends the tab by inserting a rod of the injection molding machine through the sensor port of the POD in order to apply a force to a portion of the POD (e.g., to the head portion 116 of tab 114). Bending the tab during the manufacturing process can be advantageous because the manufacturer can control the degree to which the tab is bent.

After bending the tab, first portion of the POD, the diaphragm, and the second portion of the POD are connected to one another (910). For example, the first portion of the POD is ultrasonically welded to the second portion of the POD with a portion of the diaphragm compressed between the first portion of the POD and the second portion of the POD, e.g., to form a hermetic seal along a rim of the POD. When the first portion and second portion of the POD are connected to one another, the diaphragm separates the first portion of the POD and the second portion of the POD, e.g., to separate the POD into a fluid chamber and a pressure sensing chamber.

While certain embodiments have been described, other embodiments are possible.

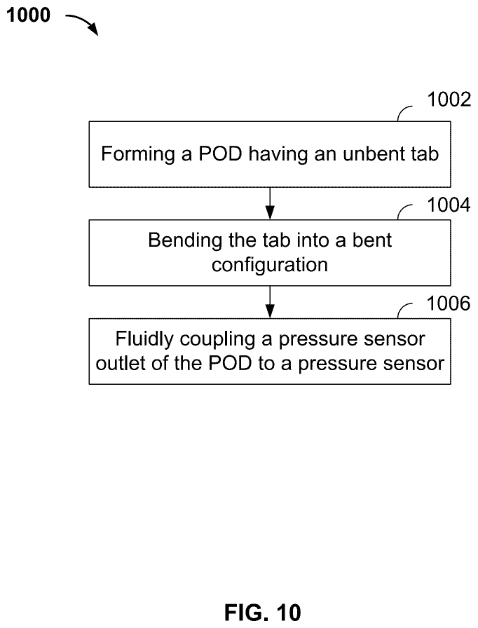

For example, while the tab 114 of the POD 100 has been described as being bent as part of the process of manufacturing the POD 100 (e.g., at a manufacturing facility), in some embodiments the tab of the POD is bent by the blood treatment machine or an operator of the blood treatment machine prior to performing a treatment using the POD. FIG. 10 illustrates a method 1000 of manufacturing a POD having an unbent tab and bending the tab for use in a blood treatment machine, such as a dialysis machine. For example, in some implementations, a user receives a POD with an unbent tab, and the tab is bent just prior to use of the POD. Bending the tab of the POD at the location of treatment shortly before treatment, rather than during manufacturing of the POD, can be advantageous, for example, to prevent breaking or unplanned bending of the tab during shipping of the POD.

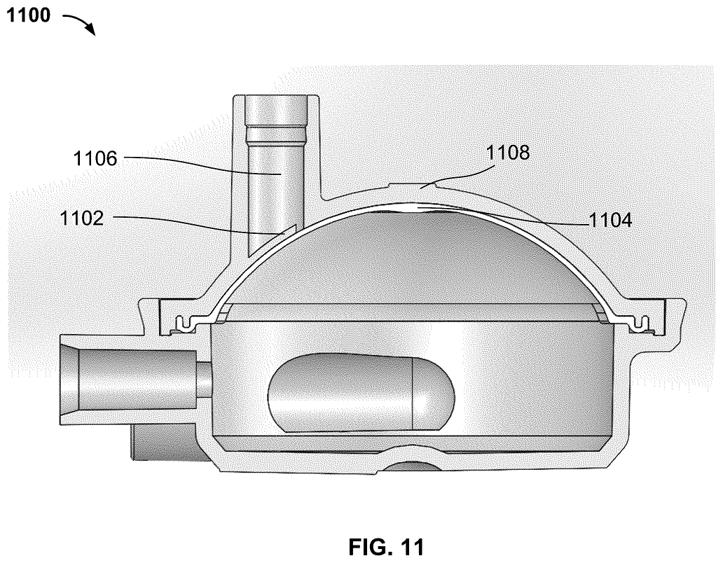

FIG. 10 is a flowchart of an example method 1000 of manufacturing a POD having a tab that is unbent (e.g., similar to the POD 1100 of FIG. 11) and coupling the POD to a pressure sensor. A POD is formed with a unbent tab (1002). For example, the POD can be formed using a method that is similar to method 900 described above but that does not include a step of bending the tab and the first and second portions of the POD are connected to one another without bending the tab of the POD. For example, the first portion of the POD, the diaphragm of the POD, and the second portion of the POD can be manufactured as described above with reference to FIG. 9, and the pieces of the POD can be attached to one another as described above, but the tab of the POD is not bent during the manufacturing process.

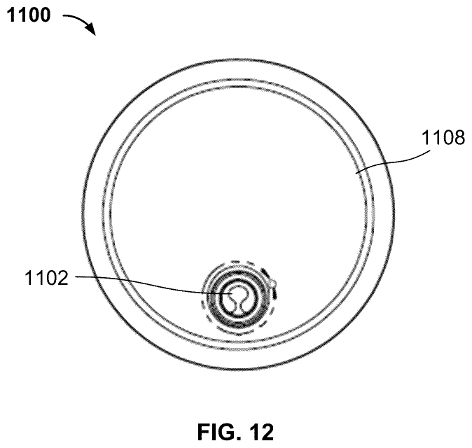

FIGS. 11 and 12 illustrate a POD 1100 manufactured via the method 1000 of FIG. 10. FIG. 11 illustrates a cross-section of the POD 1100, and FIG. 12 illustrates a top view of the POD 1100. The POD 1100 has a tab 1102 that is unbent, a diaphragm 1104, a sensor port 1106, and a cap 1108. Before using the POD 1100 during a blood treatment, the tab 1102 of the POD 1100 is bent, either by a user manually bending the tab 1102 (e.g., by inserting a rod through the sensor port 1106 of the POD 1100) or by the blood treatment device automatically bending the tab 1102 following connection of the POD 1100 to the blood treatment machine, as will be described in further detail herein.

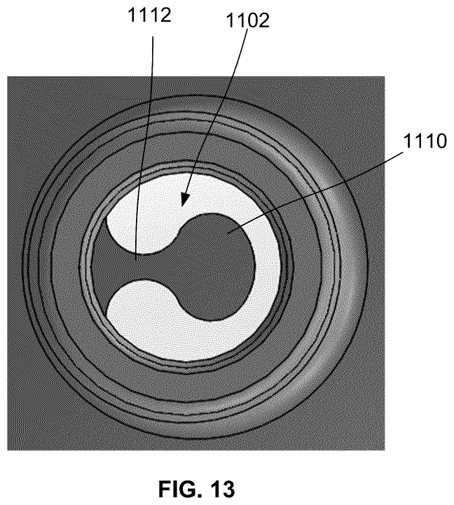

FIG. 13 is a schematic top view of the tab 1102 of the POD 1100 during manufacturing prior to being bent (e.g., by a blood treatment machine or an operator of the blood treatment machine). Similar to tab 114 of FIGS. 3 and 4, the tab 1102 of the POD 1100 includes a head portion 1110 that is wider than a hinge portion 1112 of the tab 1102 that connects the head portion 1110 to the cap 1108 of the POD 1100. The head portion 1110 of the tab 1102 is generally elliptical. Similar to the hinge portion 118 of the tab 114, the hinge portion 1112 of the tab 1102 is deformable so that the hinge portion 1112 bends relative to the cap 1108 to allow the head portion 1110 of the tab 1102 to be angled downward towards the diaphragm 1104 of the POD 1100. In some implementations, the hinge portion 1112 has a smaller width than the head portion 1110 so that the hinge portion 1112 deforms when the tab 1102 is bent.

Prior to performing blood treatment using the POD 1100, the tab 1102 of the POD 1100 is bent into a bent configuration (1004). For example, a user (e.g., an operator of a blood treatment machine) manually bends the tab of the POD by inserting a rod through the sensor port of the POD. An appropriate rod can be provided with the fully manufactured POD, and instructions can be provided on how to bend the tab properly using the rod.

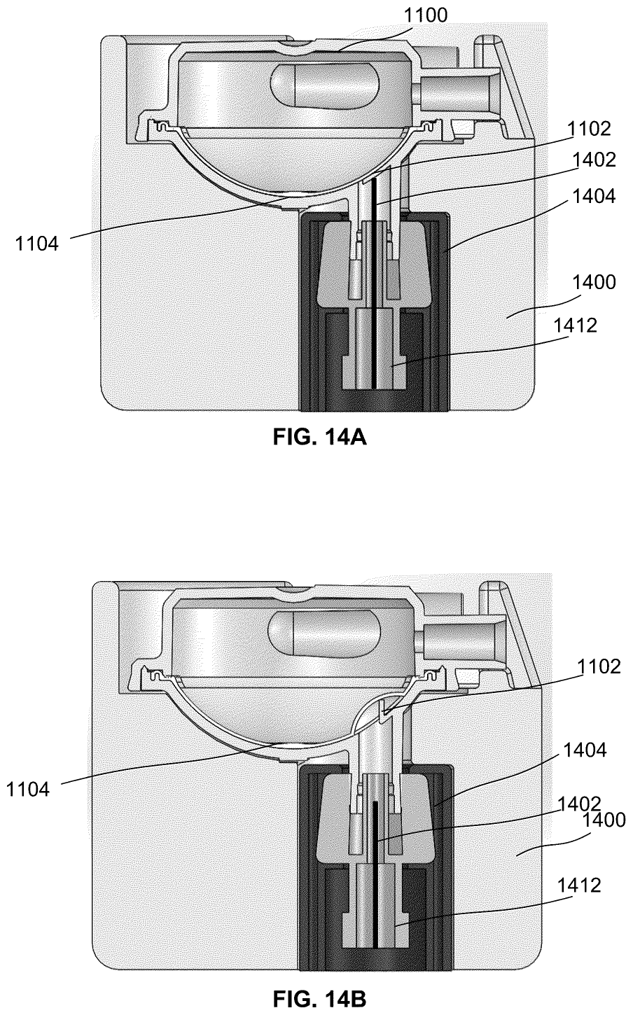

In some implementations, a portion of the blood treatment machine automatically bends the tab upon coupling the POD to the blood treatment machine. FIGS. 14A and 14B are schematic cross-sectional views of the POD 1100 connected to a blood treatment machine 1400. In some implementations, the blood treatment machine 1400 automatically bends the tab upon receiving the POD 1100. For example, similar to POD 100 in FIG. 7, POD 1100 is coupled the blood treatment machine 1400 by inserting the sensor port 1106 of the POD 1100 into a pressure monitoring port 1404 of the blood treatment machine 1400. As illustrated in FIG. 14A, the POD 1100 is coupled to the blood treatment machine 1400 with the tab in an unbent position. Following insertion of the sensor port 1106 of the POD 1100 into a pressure monitoring port 1404 of the blood treatment machine 1400, the blood treatment machine 1400 extends a rod 1402 through the pressure monitoring port 1404 and into the sensor port 1106 of the POD 1100 to apply a force to the tab 1102, which causes the tab 1102 to bend towards the diaphragm 1104 of the POD 1100 into a bent position, as depicted in FIG. 14 B. After bending the tab 1102, the blood treatment machine 1400 can cause the rod 1402 to retract inwards. FIG. 14B illustrates the retracted rod 1402 and the bent tab 1102. Similar to tab 114 of POD 100, the tab 1102 prevents the diaphragm 1104 from forming a seal with the sensor port 1106 by deforming the diaphragm 1104 away from the sensor port 1106 when the diaphragm is against the cap 1108 of the POD 1100 (e.g., as a result of high pressure within a fluid chamber of the POD 1100).

In some implementations, during priming of the blood treatment machine 1400 and treatment, the blood treatment machine 1400 can detect whether the POD 1100 is coupled to the pressure monitoring port 1404, and can generate an alarm in response to detecting a disconnection of the POD 1100 from the pressure monitoring port 1404. For example, if during priming or treatment, the pressure sensor associated with the POD 1100 does not measure any pressure in through the pressure monitoring port 1404, the blood treatment machine 1400 can generate an alarm indicating that the POD 1100 is not connected to the blood treatment machine 1400. Conversely, if the pressure sensor detects a change in pressure in the POD 1100 through the pressure monitoring port 1404, then the blood treatment machine 1400 can determine that the POD 1100 is connected to the pressure monitoring port 1404.

While the blood treatment machine 1400 has been described as extending the rod 1402 automatically, in some implementations, a user prompt or command by provided by an operator of the blood treatment machine 1400 (e.g., using a user interface of the blood treatment machine 1400), causes the rod 1402 to extend and bend the tab 1102 in response to receiving the user prompt or command. For example, a user or operator can place the POD 1100 into the pressure monitoring port 1404 and then prompt the blood treatment machine 1400 to bend the tab 1102 by providing confirmation of the attachment of the POD 1100 to the blood treatment machine 1400 through a user interface of the blood treatment machine 1400.

After bending the tab 1102 of the POD 1100 into a bent configuration, the POD 1100 is fluidly coupled to a pressure sensor, e.g., of a blood treatment machine (1006). For example, a pressure monitoring port of a blood treatment machine can fluidly connect a pressure sensor to the POD 1100, which enables the pressure sensor of the blood treatment machine 1400 to detect the pressure within the POD 1100, as described above in reference to FIG. 7. In some implementations, a user manually bends the tab 1102 (e.g., by inserting a rod through the sensor port 1106 of the POD 1100) prior to connecting the POD 1100 to the blood treatment machine 1400. The user then connects the POD 1100 to the blood treatment machine 1400 to fluidly couple the POD 1100 to the pressure sensor 1412 of the blood treatment machine 1400. In other implementations, the user connects the POD 1100 to the blood treatment machine 1400 without bending the tab 1102, and the machine bends the tab 1102 to fluidly connect the POD 1100 to the pressure sensor. For example, following insertion of the sensor port 1106 of the POD 1100 into a pressure monitoring port 1404 of the blood treatment machine 1400, the blood treatment machine 1400 extends a rod 1402 through the pressure monitoring port 1404 and into the sensor port 1106 of the POD 1100 to apply a force to the tab 1102, which causes the tab 1102 to bend towards the diaphragm 1104 of the POD 1100 into a bent position, as depicted in FIG. 14B. Once the tab 1102 of the POD 1100 is bent and the rod 1402 is retracted, the POD 1100 is fluidly coupled to the POD 1100 to the pressure sensor 1412 of the blood treatment machine 1400.

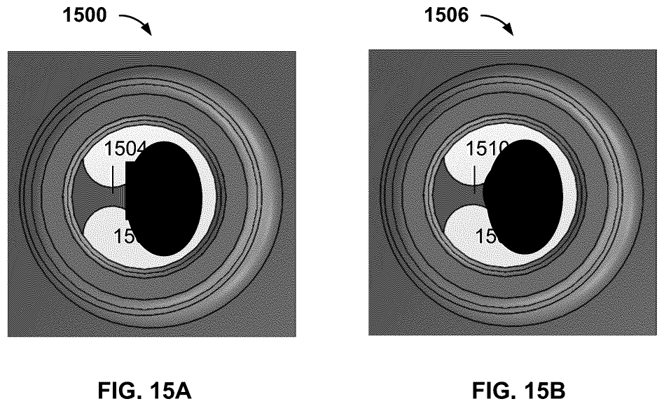

While the tab 114, 1102 of the POD 100, 1100 has been described as having a head portion 116, 1110 with an elliptical shape, the head of the tab of the POD can be other shapes. For example, FIGS. 15A and 15B illustrate different tabs that can be included in POD's similar to those described above. FIG. 15A illustrates an example tab 1500 having a head portion 1502 that is rectangular, and a hinge portion 1504 with a smaller width than the width of the rectangular head portion 1502. In some implementations, the head portion 1502 of the tab 1500 is a square shape. FIG. 15B illustrates another example tab 1506. The tab 1506 has a head portion 1508 that is circular and a hinge portion 1510 with a smaller width than the head portion 1508. Differently shaped tabs can create differently shaped dimples in the diaphragm. For example, rounded edges of a circular head portion 1508 create a differently shaped dimple than the rectangular head portion 1502. Some tab geometries require less materials to manufacture, thus making some tab geometries more cost effective.

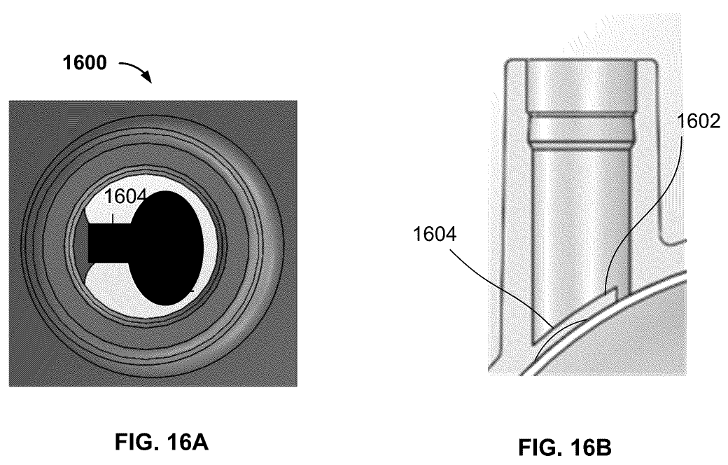

In addition, while the tab 114, 1102 of the POD 100, 1100 has been described as having a hinge portion 118, 1112 of the tab 114, 1102 with smaller width than the head portion 116, 1110 of the tab 114, 1102, in some implementations, the width of the hinge portion of the tab is the same as the width of the head portion of the hinge. FIGS. 16A and 16B illustrate a tab 1600 having a head portion 1602 and a hinge portion 1604 having the same width as the head portion 1602. Rather than having a smaller width than the head portion 1602, the hinge portion 1604 of the tab 1600 is thinner in cross-section than the head portion 1602, which causes the tab 1600 to bend about the hinge portion 1604 of the tab 1600 when a force is applied to the head portion 1602 of the tab 1600. For example, FIG. 16B illustrates a cross-section of the tab 1600, illustrating that the hinge portion 1604 is thinner than the head portion 1602. Because the hinge portion 1604 is thinner and has less material than the head portion 1602, the hinge portion 1604 is more deformable than the head portion 1602. As a result, the tab 1600 bends about the hinge portion 1604 of the tab 1600 when a force is applied to the head portion 1602 of the tab 1600. In some implementations, the hinge portion of a tab can be thinner in cross-section than the head portion of the tab and have a smaller width than the head portion of the tab.

While the POD has been described as being part of a hemodialysis system during hemodialysis treatment, the POD can also be used in any of various other types of medical fluid pumping systems including, but not limited to, peritoneal dialysis treatment, hemofiltration treatment, and hemodiafiltration treatment.

A number of embodiments have been described. Nevertheless, it will be understood that various modifications may be made without departing from the spirit and scope of the disclosure. Accordingly, other embodiments are within the scope of the following claims.

What is claimed is:

1. A pressure output device (POD) for sensing fluid pressure in a blood line set, the POD comprising:
a base comprising an inlet port and an outlet port;
a cap coupled to the base to form an interior chamber of the POD, the cap comprising:
an outer shell;
a pressure sensor port extending from the outer shell; and
a tab extending inwardly from an inner surface of the outer shell;
a flexible diaphragm coupled to the cap and positioned over the base to divide the interior chamber into a fluid chamber and a pressure sensing chamber, the tab configured to deform a portion of the flexible diaphragm.

2. The POD of claim 1, wherein the tab is positioned beneath the pressure sensor port.

3. The POD of claim 1, wherein the tab is configured to prevent the flexible diaphragm from covering an opening of the pressure sensor port.

4. The POD of claim 1, wherein the cap and the flexible diaphragm are formed using a two shot molding process.

5. The POD of claim 1, wherein the tab comprises:
a first portion configured to contact the flexible diaphragm; and
a second portion configured to bend to adjust a position of the first portion.

6. The POD of claim 5, wherein a width of the first portion is larger than a width of the second portion.

7. The POD of claim 5, wherein a thickness of the first portion is larger than a thickness of the second portion.

8. The POD of claim 5, wherein the first portion has a cross-sectional shape that is circular, rectangular, elliptical, or square.

9. The POD of claim 1, wherein the tab elastically dimples the flexible diaphragm when the tab is in a bent position and the flexible diaphragm contacts the tab.

10. The POD of claim 1, wherein the base and the outer shell of the cap are semi-rigid.

11. The POD of claim 1, wherein the base and the outer shell of the cap are translucent.

12. The POD of claim 1, wherein the tab is configured to be bent into a bent position prior to a blood treatment performed using the blood line set.

13. A method of assembling a pressure output device (POD), the method comprising:
bending a tab of a first portion of the POD into a bent configuration in which the tab is configured to form a dimple in a portion of a flexible diaphragm when the flexible diaphragm is in contact with the tab, the portion of the flexible diaphragm being positioned below a pressure sensor outlet of the POD; and
connecting the first portion of the POD, the flexible diaphragm, and a second portion of the POD.

14. The method of claim 13, further comprising forming the first portion using a first shot of a two-shot molding process, and forming the flexible diaphragm using a second shot of the two-shot molding process.

15. The method of claim 13, wherein bending the tab comprises bending the tab along a first portion of the tab that is thinner or has a smaller width than a second portion of the tab.

16. The method of claim 13, wherein bending the tab comprises bending the tab by inserting a rod through a port of the POD and applying a force to the tab.

17. A blood treatment system, comprising:
a blood treatment machine;
a blood line set configured to be connected to the blood treatment machine; and
a pressure output device (POD) for sensing fluid pressure in the blood line set, the POD comprising:
a base comprising an inlet port and an outlet port;
a cap coupled to the base to form an interior chamber of the POD, the cap comprising:
an outer shell;
a pressure sensor port extending from the outer shell; and
a tab extending inwardly from an inner surface of the outer shell;
a flexible diaphragm coupled to the cap and positioned over the base to divide the interior chamber into a fluid chamber and a pressure sensing chamber, the tab configured to deform a portion of the flexible diaphragm.

18. The blood treatment system of claim 17, wherein the blood treatment machine is a dialysis machine.

19. The blood treatment system of claim 18, wherein:
the blood treatment machine is a hemodialysis machine; and
the blood treatment system further comprises a dialyzer.

20. The blood treatment system of claim 17, wherein the blood line set comprises at least one of an arterial line and a venous line.

21. The blood treatment system of claim 17, wherein the tab is configured to be bent into a bent position by a pin of the blood treatment machine that extends through the pressure sensor port and contacts the tab when the POD is coupled to the blood treatment machine.
